(19) United States
(12) Patent Application Publication
(10) Pub. No.: US 2007/0178099 A1
(43) Pub. Date: Aug. 2, 2007

(54) Suppression of TNFalpha and IL-12 in therapy

(75) Inventors: Marc Feldmann, London (GB); Anne-Marie Malfait, London (GB); Debra M. Butler, Surrey (GB); Fionula M. Brennan, London (GB); Ravinder N. Maini, London (GB)

Correspondence Address: COOPER & DUNHAM, LLP, 1185 AVENUE OF THE AMERICAS, NEW YORK, NY, 10036, US

(73) Assignee: The Kennedy Institute of Rheumatology

(21) Appl. No.: 11/728,661

(22) Filed: Mar. 26, 2007

Related U.S. Application Data
(63) Continuation of application No. 08/749,979, filed on Nov. 15, 1996, now abandoned.

(51) Int. Cl. A61K 39/395; A61K 31/522; A61K 31/454

(52) U.S. Cl. 424/145.1; 514/263.31; 514/323

(57) ABSTRACT

Methods for treating and/or preventing a TNF-mediated disease in an individual are disclosed. Also disclosed are compositions comprising a TNF antagonist and an IL-12 antagonist. TNF-mediated diseases include rheumatoid arthritis, Crohn's disease, and acute and chronic immune diseases associated with transplantation.

### BACKGROUND OF THE INVENTION

Monocytes and macrophages secrete cytokines known as tumor necrosis factor alpha (TNFα), interleukin-1 (IL-1) and interleukin-6 (IL-6) in response to endotoxin or other stimuli. TNFα is a soluble homotrimer of 17 kD protein subunits (Smith et al., J. Biol. Chem. 262:6951-6954 (1987)). A membrane-bound 26 kD precursor form of TNF also exists (Kriegler et al., Cell 53:45-53 (1988)). For reviews of TNF, see Beutler et al., Nature 320:584 (1986); Old, Science 230:630 (1986); and Le et al., Lab. Invest. 56:234 (1987).

Cells other than monocytes or macrophages also produce TNFα. For example, human non-monocytic tumor cell lines produce tumor necrosis factor (TNF) (Rubin et al., J. Exp. Med. 164:1350 (1986); Spriggs et al., Proc. Natl. Acad. Sci. USA 84:6563 (1987)). CD4+ and CD8+ peripheral blood T lymphocytes and some cultured T and B cell lines (Cuturi et al., J. Exp. Med. 165:1581 (1987); Sung et al., J. Exp. Med. 168:1539 (1988); Turner et al., Eur. J. Immunol. 17:1807-1814 (1987)) also produce TNFα.

TNF causes pro-inflammatory actions which result in tissue injury, such as degradation of cartilage and bone (Saklatvala, Nature 322:547-549 (1986); Bertolini, Nature 319:516-518 (1986)), induction of adhesion molecules, inducing procoagulant activity on vascular endothelial cells (Pober et al., J. Immunol. 136:1680 (1986)), increasing the adherence of neutrophils and lymphocytes (Pober et al., J. Immunol. 138:3319 (1987)), and stimulating the release of platelet activating factor from macrophages, neutrophils and vascular endothelial cells (Camussi et al., J. Exp. Med. 166:1390 (1987)).

Recent evidence associates TNF with infections (Cerami et al., Immunol. Today 9:28 (1988)), immune disorders, neoplastic pathologies (Oliff et al., Cell 50:555 (1987)), autoimmune pathologies and graft-versus-host pathologies (Piguet et al., J. Exp. Med. 166:1280 (1987)). The association of TNF with cancer and infectious pathologies is often related to the host's catabolic state. Cancer patients suffer from weight loss, usually associated with anorexia.

The extensive wasting which is associated with cancer, and other diseases, is known as “cachexia” (Kern et al., J. Parent. Enter. Nutr. 12:286-298 (1988)). Cachexia includes progressive weight loss, anorexia, and persistent erosion of lean body mass in response to a malignant growth. The cachectic state causes much cancer morbidity and mortality. There is evidence that TNF is involved in cachexia in cancer, infectious pathology, and other catabolic states (see, e.g., Beutler and Cerami, Ann. Rev. Immunol. 7:625-655 (1989)).

TNF is believed to play a central role in gram-negative sepsis and endotoxic shock (Michie et al., Br. J. Surg. 76:670-671 (1989); Debets et al., Second Vienna Shock Forum, p. 463-466 (1989); Simpson et al., Crit. Care Clin. 5:27-47 (1989)), including fever, malaise, anorexia, and cachexia. Endotoxin strongly activates monocyte/macrophage production and secretion of TNF and other cytokines (Kornbluth et al., J. Immunol. 137:2585-2591 (1986)). TNF and other monocyte-derived cytokines mediate the metabolic and neurohormonal responses to endotoxin (Michie et al., New Engl. J. Med. 318:1481-1486 (1988)). Endotoxin administration to human volunteers produces acute illness with flu-like symptoms including fever, tachycardia, increased metabolic rate and stress hormone release (Revhaug et al., Arch. Surg. 123:162-170 (1988)). Circulating TNF increases in patients suffering from Gram-negative sepsis (Waage et al., Lancet 1:355-357 (1987); Hammerle et al., Second Vienna Shock Forum, p. 715-718 (1989); Debets et al., Crit. Care Med. 17:489-497 (1989); Calandra et al., J. Infect. Dis. 161:982-987 (1990)).

Thus, TNFα has been implicated in inflammatory diseases, autoimmune diseases, viral, bacterial and parasitic infections, malignancies, and/or neurogenerative diseases and is a useful target for specific biological therapy in diseases, such as rheumatoid arthritis and Crohn's disease. Beneficial effects in open-label trials with a chimeric monoclonal antibody to TNFα (cA2) have been reported with suppression of inflammation and with successful retreatment after relapse in rheumatoid arthritis (Elliott et al., Arthritis Rheum. 36:1681-1690 (1993); and Elliott et al., Lancet 344:1125-1127 (1994)) and in Crohn's disease (Van Dullemen et al., Gastroenterology 109:129-135 (1995)). Beneficial results in a randomized, double-blind, placebo-controlled trial with cA2 have also been reported in rheumatoid arthritis with suppression of inflammation (Elliott et al., Lancet 344:1105-1110 (1994)).

Monocytes, macrophages and other antigen-presenting cells, such as dendritic cells, also secrete a cytokine known as interleukin-12 (IL-12) in response to bacterial products and immunological stimuli. IL-12 is a heterodimeric cytokine, consisting of a p40 and a p35 subunit, with potent immunoregulatory properties (reviewed in Trinchieri, Annu. Rev. Immunol. 13:251-276 (1995); and Trinchieri, Blood 84:4008-4027 (1994)). IL-12 enhances natural killer (NK)-mediated cytotoxicity and induces interferon γ (IFN-γ) production by NK cells and T lymphocytes (Wolf et al., J. Immunol. 146:3074-3081 (1991); and Chan et al., J. Exp. Med. 173:869-879 (1991)). IL-12 plays a key role in promoting T helper type 1 (Th1) immune responses in vitro (Manetti et al., J. Exp. Med. 177:1199-1204 (1993)) and in vivo (Sypek et al., J. Exp. Med. 177:1797-1802 (1993); and Heinzel et al., J. Exp. Med. 177:1505-1509 (1993)).

IL-12 has been shown to accelerate the onset of autoimmune diabetes, a Th1-mediated disease, in nonobese diabetic (NOD) mice (Trembleau et al., J. Exp. Med. 181:817-821 (1995)). It has also been demonstrated that IL-12 can replace Mycobacterium tuberculosis when immunizing DBA/1 mice with type II collagen in oil to profoundly upregulate a Th1-type autoimmune response, resulting in arthritis (Germann et al., Proc. Natl. Acad. Sci. USA 92:4823-4827 (1995)).

Antibodies against IL-12 have been shown to be beneficial in experimental models for autoimmune diseases that are Th1 driven, such as experimental allergic encephalomyelitis (EAE) (Leonard et al., J. Exp. Med. 181:381-386 (1995)) and 2,4,6-trinitrobenzene sulfonic acid (TNBS)-induced chronic intestinal inflammation in mice, a model for human inflammatory bowel disease (Neurath et al., J. Exp. Med. 182:1281-1290 (1995)). However, paradoxical effects of IL-12 in experimental models have been described. For example, very high doses of IL-12 administered in the induction phase of type II collagen induced arthritis (CIA) have been reported to suppress the immune response and prevent the onset of arthritis (Hess et al., Eur. J. Immunol. 26:187-191 (1996)). Similarly, although continuous administration of IL-12 accelerates the onset of diabetes in NOD mice, intermittent administration completely prevents the diabetes (Trembleau et al., Immunol. Today 16:383-387 (1995)).

The role of Th1/Th2-type responses in the development of CIA has been investigated by measuring IFN-γ and IL-4/IL-10 production in type II collagen (CII)-stimulated draining lymph node cells cultured at different stages of the disease (Mauri et al., Eur. J. Imm. 26:1511-1518 (1996)). It was found that IFN-γ production dramatically increased at the time of disease onset and subsequently declined throughout the disease and the remission phase, in favor of the Th2 cytokines IL-4 and IL-10.

The association between Th1 CD4+ T cell responses and the onset of arthritis suggests that IL-12 blockade should be beneficial in CIA. Indeed, IL-12p40 knockout mice displayed a dramatic decrease in severity of CIA.

However, in adult male DBA/1 mice, the effects of neutralizing IL-12 with anti-IL-12 antibodies in the induction phase of the disease were dependent on the treatment protocol. Prolonged blockade of IL-12 from immunization until the onset of disease dramatically attenuated the severity of the arthritis. However, when IL-12 was blocked immediately after immunization for only a short period (i.e. 2 weeks), a dichotomous response was observed. Half of the mice developed a very mild arthritis or no disease; the other half developed an unusually severe arthritis. Neutralizing anti-IL-12 antibodies administered after disease onset had minimal, if any, effect on the course of CIA.

### SUMMARY OF THE INVENTION

The present invention is based on the unexpected discovery that co-administration of a tumor necrosis factor (TNF) antagonist and an interleukin-12 (IL-12) antagonist produces a rapid and sustained reduction in the signs and symptoms associated with TNF-mediated diseases. As a result of Applicants' invention, a method is provided herein for treating and/or preventing a TNF-mediated disease in an individual comprising co-administering a TNF antagonist and an IL-12 antagonist to the individual in therapeutically effective amounts. The present invention further relates to a method for treating and/or preventing recurrence of a TNF-mediated disease in an individual comprising co-administering a TNF antagonist and an IL-12 antagonist to the individual in therapeutically effective amounts. TNF-mediated diseases include rheumatoid arthritis, Crohn's disease, and acute and chronic immune diseases associated with an allogenic transplantation (e.g., renal, cardiac, bone marrow, liver, pancreatic, small intestine, skin or lung transplantation).

Therefore, in one embodiment, the invention relates to a method of treating and/or preventing (such as preventing relapse of) rheumatoid arthritis in an individual comprising co-administering a TNF antagonist and an IL-12 antagonist to the individual in therapeutically effective amounts. In a second embodiment, the invention relates to a method of treating and/or preventing (such as preventing relapse of) Crohn's disease in an individual comprising co-administering a TNF antagonist and an IL-12 antagonist to the individual in therapeutically effective amounts. In a third embodiment, the invention relates to a method of treating and/or preventing acute or chronic immune disease associated with a transplantation in an individual comprising co-administering a TNF antagonist and an IL-12 antagonist to the individual in therapeutically effective amounts.

A further embodiment of the invention relates to compositions comprising a TNF antagonist and an IL-12 antagonist.

TNF antagonists useful in the methods and compositions of the present invention include anti-TNF antibodies and receptor molecules which bind specifically to TNF; compounds which prevent and/or inhibit TNF synthesis, TNF release or its action on target cells, such as thalidomide, tenidap, phosphodiesterase inhibitors (e.g., pentoxifylline and rolipram), A2b adenosine receptor agonists and A2b adenosine receptor enhancers; compounds which prevent and/or inhibit TNF receptor signalling, such as mitogen activated protein (MAP) kinase inhibitors; compounds which block and/or inhibit membrane TNF cleavage, such as metalloproteinase inhibitors; compounds which block and/or inhibit TNF activity, such as angiotensin converting enzyme (ACE) inhibitors (e.g., captopril); and compounds which block and/or inhibit TNF production and/or synthesis, such as MAP kinase inhibitors.

IL-12 antagonists useful in the methods and compositions of the present invention include anti-IL-12 antibodies; IL-12 p40 homodimers; IL-12 p35 homodimers; receptor molecules which bind specifically to IL-12; compounds (e.g., drugs and other agents, including antibodies) which decrease and/or block IL-12 production and/or synthesis, such as β-adrenergic agonists (e.g., salbutamol); and compounds which block and/or interfere with IL-12 receptor signalling. IL-12 antagonists useful in the present invention also include agents which are antagonists of signals that drive IL-12 production and/or synthesis, such as agents which decrease and/or block CD40 or its ligand.

In a particular embodiment of the invention, an inflammatory mediator other than a TNF antagonist and/or an IL-12 antagonist can be used instead of or in addition to the TNF antagonist and/or the IL-12 antagonist.

### BRIEF DESCRIPTION OF THE DRAWINGS

FIGS. 1A-1B and 2A-2B are graphs showing the effect of administering anti-TNF antibody in combination with anti-IL-12 antibody to male DBA/1 mice on the suppression of arthritis as assessed by paw-swelling measurements (FIGS. 1B and 2A) and clinical score (FIGS. 1A and 2B). White circle =PBS control; black circle=anti-TNF antibody; square=anti-IL-12 antibody; triangle=anti-TNF antibody plus anti-IL-12 antibody.

FIGS. 3A and 3B are bar graphs showing the results of a histological analysis of arthritis within the hind paw (FIG. 3A) and knee (FIG. 3B) after treatment with anti-TNF antibody in combination with anti-IL-12 antibody.

FIGS. 4A and 4B are bar graphs showing serum levels of anti-type II collagen antibodies after treatment with anti-TNF antibody in combination with anti-IL-12 antibody.

### DETAILED DESCRIPTION OF THE INVENTION

The present invention relates to the unexpected and surprising discovery that co-administration of a TNF antagonist and an IL-12 antagonist in treating a TNF-mediated disease produces a significantly improved response compared to that obtained with administration of the TNF antagonist alone or that obtained with administration of the IL-12 antagonist alone. As a result of Applicants' invention, a method is provided herein for treating and/or preventing a TNF-mediated disease in an individual, comprising co-administering a tumor necrosis factor antagonist and an IL-12 antagonist to the individual in therapeutically effective amounts. The TNF antagonist and IL-12 antagonist can be administered simultaneously or sequentially. The anti-TNF antibody and anti-IL-12 antibody can each be administered in single or multiple doses. Multiple IL-12 antagonists and multiple TNF antagonists can be co-administered. Other therapeutic regimens and agents can be used in combination with the therapeutic co-administration of TNF antagonists and IL-12 antagonists.

Inflammatory mediators other than the described antagonists can be used instead of or in addition to these antagonists. As used herein, the term “inflammatory mediator” refers to an agent which decreases, blocks, inhibits, abrogates or interferes with pro-inflammatory mediator activity. Representative inflammatory mediators that can be used in the present invention include agents which decrease, block, inhibit, abrogate or interfere with IL-1 activity, synthesis, or receptor signalling, such as anti-IL-1 antibody, soluble IL-1R, IL-1 receptor antagonist, or other appropriate peptides and small molecules; agents which decrease, block, inhibit, abrogate or interfere with IL-6 activity, synthesis, or receptor signalling, such as anti-IL-6 antibody, anti-gp130, or other appropriate peptides and small molecules; modalities which decrease, block, inhibit, abrogate or interfere with the activity, synthesis, or receptor signalling of other pro-inflammatory mediators, such as GM-CSF and members of the chemokine IL-8 family; and cytokines with anti-inflammatory properties, such as IL-4, IL-10, IL-13, and TGFβ. In addition, other anti-inflammatory agents, such as the anti-rheumatic agent methotrexate, can be administered in conjunction with the IL-12 antagonist and/or the TNF antagonist. Inflammatory mediators and anti-inflammatory agents are also described in U.S. application Ser. No. 08/690,775 (filed Aug. 1, 1996) and U.S. application Ser. No. 08/607,419 (filed Feb. 28, 1996), which references are entirely incorporated herein by reference.

The present invention further relates to a method for treating and/or preventing recurrence of a TNF-mediated disease in an individual comprising co-administering a TNF antagonist and an IL-12 antagonist to the individual in therapeutically effective amounts.

As used herein, a “TNF-mediated disease” refers to a TNF related pathology or disease. TNF related pathologies or diseases include, but are not limited to, the following:

(A) acute and chronic immune and autoimmune pathologies, such as, but not limited to, rheumatoid arthritis (RA), juvenile chronic arthritis (JCA), spondyloarthropathy, thyroiditis, graft versus host disease (GVHD), scleroderma, diabetes mellitus, Graves' disease, allergy, acute or chronic immune disease associated with an allogenic transplantation, such as, but not limited to, renal transplantation, cardiac transplantation, bone marrow transplantation, liver transplantation, pancreatic transplantation, small intestine transplantation, lung transplantation and skin transplantation;

(B) infections, including, but not limited to, sepsis syndrome, cachexia, circulatory collapse and shock resulting from acute or chronic bacterial infection, acute and chronic parasitic and/or infectious diseases, bacterial, viral or fungal, such as a human immunodeficiency virus (HIV), acquired immunodeficiency syndrome (AIDS) (including symptoms of cachexia, autoimmune disorders, AIDS dementia complex and infections);

(C) inflammatory diseases, such as chronic inflammatory pathologies, including chronic inflammatory pathologies such as, but not limited to, sarcoidosis, chronic inflammatory bowel disease, ulcerative colitis, and Crohn's pathology or disease; vascular inflammatory pathologies, such as, but not limited to, disseminated intravascular coagulation, atherosclerosis, Kawasaki's pathology and vasculitis syndromes, such as, but not limited to, polyarteritis nodosa, Wegener's granulomatosis, Henoch-Schönlein purpura, giant cell arthritis and microscopic vasculitis of the kidneys; chronic active hepatitis; Sjögren's syndrome; spondyloarthropathies, such as ankylosing spondylitis, psoriatic arthritis and spondylitis, enteropathic arthritis and spondylitis, reactive arthritis and arthritis associated with inflammatory bowel disease; and uveitis;

(D) neurodegenerative diseases, including, but not limited to, demyelinating diseases, such as multiple sclerosis and acute transverse myelitis; myasthenia gravis; extrapyramidal and cerebellar disorders, such as lesions of the corticospinal system; disorders of the basal ganglia or cerebellar disorders; hyperkinetic movement disorders, such as Huntington's chorea and senile chorea; drug-induced movement disorders, such as those induced by drugs which block central nervous system (CNS) dopamine receptors; hypokinetic movement disorders, such as Parkinson's disease; progressive supranuclear palsy; cerebellar and spinocerebellar disorders, such as astructural lesions of the cerebellum; spinocerebellar degenerations (spinal ataxia, Friedreich's ataxia, cerebellar cortical degenerations, multiple systems degenerations (Mencel, Dejerine-Thomas, Shi-Drager, and MachadoJoseph)); and systemic disorders (Refsum's disease, abetalipoproteinemia, ataxia, telangiectasia, and mitochondrial multisystem disorder); disorders of the motor unit, such as neurogenic muscular atrophies (anterior horn cell degeneration, such as amyotrophic lateral sclerosis, infantile spinal muscular atrophy and juvenile spinal muscular atrophy); Alzheimer's disease; Down's syndrome in middle age; diffuse Lewy body disease; senile dementia of Lewy body type; Wernicke-Korsakoff syndrome; chronic alcoholism; primary biliary cirrhosis; cryptogenic fibrosing alveolitis and other fibrotic lung diseases; hemolytic anemia; Creutzfeldt-Jakob disease; subacute sclerosing panencephalitis, Hallerrorden-Spatz disease; and dementia pugilistica, or any subset thereof;

(E) malignant pathologies involving TNF-secreting tumors or other malignancies involving TNF, such as, but not limited to, leukemias (acute, chronic myelocytic, chronic lymphocytic and/or myelodyspastic syndrome); lymphomas (Hodgkin's and non-Hodgkin's lymphomas, such as malignant lymphomas (Burkitt's lymphoma or Mycosis fungoides));

(F) cachectic syndromes and other pathologies and diseases involving excess TNF, such as, but not limited to, cachexia of cancer, parasitic disease and heart failure; and

(G) alcohol-induced hepatitis and other forms of chronic hepatitis.

See, e.g., Berkow et al., Eds., The Merck Manual, 16th edition, chapter 11, pp. 1380-1529, Merck and Co., Rahway, N.J., 1992, incorporated herein by reference.

The terms “recurrence”, “flare-up” or “relapse” are defined to encompass the reappearance of one or more symptoms of the disease state. For example, in the case of rheumatoid arthritis, a recurrence can include the experience of one or more of swollen joints, morning stiffness or joint tenderness.

In one embodiment, the invention relates to a method of treating and/or preventing rheumatoid arthritis in an individual comprising co-administering a TNF antagonist and an IL-12 antagonist to the individual in therapeutically effective amounts.

In a second embodiment, the invention relates to a method for treating and/or preventing Crohn's disease in an individual comprising co-administering a TNF antagonist and an IL-12 antagonist to the individual in therapeutically effective amounts.

In a third embodiment, the invention relates to a method for treating and/or preventing an acute or chronic immune disease associated with an allogenic transplantation in an individual comprising co-administering a TNF antagonist and an IL-12 antagonist to the individual in therapeutically effective amounts. As used herein, a “transplantation” includes organ, tissue or cell transplantation, such as renal transplantation, cardiac transplantation, bone marrow transplantation, liver transplantation, pancreatic transplantation, small intestine transplantation, skin transplantation and lung transplantation.

The benefits of combination therapy with TNF antagonists and IL-12 antagonists are significantly improved clinical response. A rapid and sustained reduction in the clinical signs and symptoms of the disease is possible. In addition, lower dosages can be used to provide the same reduction of the immune and inflammatory response, thus increasing the therapeutic window between a therapeutic and a toxic effect. Lower doses also result in lower financial costs to the patient, and potentially fewer side effects. For example, immune and/or allergic responses to TNF antagonists can be reduced, thus enhancing safety and therapeutic efficacy.

In a further embodiment, the invention relates to compositions comprising a TNF antagonist and an IL-12 antagonist. The compositions of the present invention are useful for treating a subject having a pathology or condition associated with abnormal levels of a substance reactive with a TNF antagonist, in particular TNF in excess of, or less than, levels present in a normal healthy subject, where such excess or diminished levels occur in a systemic, localized or particular tissue type or location in the body. Such tissue types can include, but are not limited to, blood, lymph, central nervous system (CNS), liver, kidney, spleen, heart muscle or blood vessels, brain or spinal cord white matter or grey matter, cartilage, ligaments, tendons, lung, pancreas, ovary, testes, prostate. Increased or decreased TNF concentrations relative to normal levels can also be localized to specific regions or cells in the body, such as joints, nerve blood vessel junctions, bones, specific tendons or ligaments, or sites of infection, such as bacterial or viral infections.

Tumor Necrosis Factor Antagonists

As used herein, a “tumor necrosis factor antagonist” decreases, blocks, inhibits, abrogates or interferes with TNF activity in vivo. For example, a suitable TNF antagonist can bind TNF and includes anti-TNF antibodies and receptor molecules which bind specifically to TNF. A suitable TNF antagonist can also prevent or inhibit TNF synthesis and/or TNF release and includes compounds such as thalidomide, tenidap, and phosphodiesterase inhibitors, such as, but not limited to, pentoxifylline and rolipram. A suitable TNF antagonist that can prevent or inhibit TNF synthesis and/or TNF release also includes A2b adenosine receptor enhancers and A2b adenosine receptor agonists (e.g., 5′-(N-cyclopropyl)-carboxamidoadenosine, 5′-N-ethylcarboxamidoadenosine, cyclohexyladenosine and R—N⁶-phenyl-2-propyladenosine). See, for example, Jacobson (GB 2 289 218 A), the teachings of which are entirely incorporated herein by reference. A suitable TNF antagonist can also prevent or inhibit TNF receptor signalling and includes mitogen activated protein (MAP) kinase inhibitors (e.g., SB 203580; Lee and Young, J. Leukocyte Biol. 59:152-157 (1996), the teachings of which are entirely incorporated herein by reference). Other suitable TNF antagonists include agents which decrease, block, inhibit, abrogate or interfere with membrane TNF cleavage, such as, but not limited to, metalloproteinase inhibitors; agents which decrease, block, inhibit, abrogate or interfere with TNF activity, such as, but not limited to, angiotensin converting enzyme (ACE) inhibitors, such as captopril, enalapril and lisinopril; and agents which decrease, block, inhibit, abrogate or interfere with TNF production and/or synthesis, such as, but not limited to MAP kinase inhibitors. TNF antagonists are also described in U.S. application Ser. No. 08/690,775 (filed Aug. 1, 1996), U.S. application Ser. No. 08/607,419 (filed Feb. 28, 1996), International Publication No. WO 95/09652 (published Apr. 13, 1995), U.S. application Ser. No. 08/403,785 (filed Oct. 6, 1993), International Publication No. WO 94/08619 (published Apr. 28, 1994), U.S. application Ser. No. 07/958,248 (filed Oct. 8, 1992). These references are all entirely incorporated herein by reference.

Anti-TNF Antibodies

As used herein, an “anti-tumor necrosis factor antibody” decreases, blocks, inhibits, abrogates or interferes with TNF activity in vivo. Anti-TNF antibodies useful in the methods and compositions of the present invention include monoclonal, chimeric, humanized, resurfaced and recombinant antibodies and fragments thereof which are characterized by high affinity binding to TNF and low toxicity (including human anti-murine antibody (HAMA) and/or human anti-chimeric antibody (HACA) response). In particular, an antibody where the individual components, such as the variable region, constant region and framework, individually and/or collectively possess low immunogenicity is useful in the present invention. The antibodies which can be used in the invention are characterized by their ability to treat patients for extended periods with good to excellent alleviation of symptoms and low toxicity. Low immunogenicity and/or high affinity, as well as other undefined properties, may contribute to the therapeutic results achieved.

An example of a monoclonal antibody useful in the methods and compositions of the present invention is murine monoclonal antibody (mAb) A2 and antibodies which will competitively inhibit in vivo the binding to human TNFα of anti-TNFα murine mAb A2 or an antibody having substantially the same specific binding characteristics, as well as fragments and regions thereof. Murine monoclonal antibody A2 and chimeric derivatives thereof, such as cA2, are described in U.S. application Ser. No. 08/192,093 (filed Feb. 4, 1994), U.S. application Ser. No. 08/192,102 (filed Feb. 4, 1994), U.S. application Ser. No. 08/192,861 (filed Feb. 4, 1994), U.S. application Ser. No. 08/324,799 (filed Oct. 18, 1994), and Le, J. et al., International Publication No. WO 92/16553 (published Oct. 1, 1992), which references are entirely incorporated herein by reference. Preferred methods for determining mAb specificity and affinity by competitive inhibition can be found in Harlow, et al., Antibodies: A Laboratory Manual, Cold Spring Harbor Laboratory Press, Cold Spring Harbor, N.Y. (1988); Colligan et al., eds., Current Protocols in Immunology, Greene Publishing Assoc. and Wiley Interscience, New York (1992, 1993); Kozbor et al., Immunol. Today 4:72-79 (1983); Ausubel et al., eds., Current Protocols in Molecular Biology, Wiley Interscience, New York (1987, 1992, 1993); and Muller, Meth. Enzymol. 92:589-601 (1983), which references are entirely incorporated herein by reference.

Additional examples of monoclonal anti-TNF antibodies that can be used in the present invention are described in the art (see, e.g., U.S. Pat. No. 5,231,024; Möller, A. et al., Cytokine 2(3):162-169 (1990); U.S. application Ser. No. 07/943,852 (filed Sep. 11, 1992); Rathjen et al., International Publication No. WO 91/02078 (published Feb. 21, 1991); Rubin et al., EPO Patent Publication No. 0 218 868 (published Apr. 22, 1987); Yone et al., EPO Patent Publication No. 0 288 088 (Oct. 26, 1988); Liang, et al., Biochem. Biophys. Res. Comm. 137:847-854 (1986); Meager, et al., Hybridoma 6:305-311 (1987); Fendly et al., Hybridoma 6:359-369 (1987); Bringman, et al., Hybridoma 6:489-507 (1987); and Hirai, et al., J. Immunol. Meth. 96:57-62 (1987), which references are entirely incorporated herein by reference).

Chimeric antibodies are immunoglobulin molecules characterized by two or more segments or portions derived from different animal species. Generally, the variable region of the chimeric antibody is derived from a non-human mammalian antibody, such as a murine mAb, and the immunoglobulin constant region is derived from a human immunoglobulin molecule. Preferably, a variable region with low immunogenicity is selected and combined with a human constant region which also has low immunogenicity, the combination also preferably having low immunogenicity. “Low” immunogenicity is defined herein as raising significant HACA or HAMA responses in less than about 75%, or preferably less than about 50% of the patients treated and/or raising low titres in the patient treated (less than about 300, preferably less than about 100 measured with a double antigen enzyme immunoassay) (Elliott et al., Lancet 344:1125-1127 (1994), incorporated herein by reference).

As used herein, the term “chimeric antibody” includes monovalent, divalent or polyvalent immunoglobulins. A monovalent chimeric antibody is a dimer (HL)) formed by a chimeric H chain associated through disulfide bridges with a chimeric L chain. A divalent chimeric antibody is a tetramer (H2L2) formed by two HL dimers associated through at least one disulfide bridge. A polyvalent chimeric antibody can also be produced, for example, by employing a CH region that aggregates (e.g., from an IgM H chain, or μ chain).

Antibodies comprise individual heavy (H) and/or light (L) immunoglobulin chains. A chimeric H chain comprises an antigen binding region derived from the H chain of a non-human antibody specific for TNF, which is linked to at least a portion of a human H chain C region (CH), such as CH1 or CH2. A chimeric L chain comprises an antigen binding region derived from the L chain of a non-human antibody specific for TNF, linked to at least a portion of a human L chain C region (CL).

Chimeric antibodies and methods for their production have been described in the art (Morrison et al., Proc. Natl. Acad. Sci. USA 81:6851-6855 (1984); Boulianne et al., Nature 312:643-646 (1984); Neuberger et al., Nature 314:268-270 (1985); Taniguchi et al., European Patent Application No. 171496 (published Feb. 19, 1985); Morrison et al., European Patent Application No. 173494 (published Mar. 5, 1986); Neuberger et al., PCT Application No. WO 86/01533, (published Mar. 13, 1986); Kudo et al., European Patent Application No. 184187 (published Jun. 11, 1986); Morrison et al., European Patent Application No. 173494 (published Mar. 5, 1986); Sahagan et al., J. Immunol. 137:1066-1074 (1986); Robinson et al., International Publication No. PCT/US86/02269 (published May 7, 1987); Liu et al., Proc. Natl. Acad. Sci. USA 84:3439-3443 (1987); Sun et al., Proc. Natl. Acad. Sci. USA 84:214-218 (1987); Better et al., Science 240:1041-1043 (1988); and Harlow and Lane, Antibodies: A Laboratory Manual, Cold Spring Harbor Laboratory, New York, 1988). These references are entirely incorporated herein by reference.

The anti-TNF chimeric antibody can comprise, for example, two light chains and two heavy chains, each of the chains comprising at least part of a human constant region and at least part of a variable (V) region of non-human origin having specificity to human TNF, said antibody binding with high affinity to an inhibiting and/or neutralizing epitope of human TNF, such as the antibody cA2. The antibody also includes a fragment or a derivative of such an antibody, such as one or more portions of the antibody chain, such as the heavy chain constant or variable regions, or the light chain constant or variable regions.

Humanizing and resurfacing the antibody can further reduce the immunogenicity of the antibody. See, for example, Winter (U.S. Pat. No. 5,225,539 and EP 239,400 B1), Padlan et al. (EP 519,596 A1) and Pedersen et al. (EP 592,106 A1). These references are incorporated herein by reference.

Preferred antibodies useful in the methods and compositions of the present invention are high affinity human-murine chimeric anti-TNF antibodies, and fragments or regions thereof, that have potent inhibiting and/or neutralizing activity in vivo against human TNFα. Such antibodies and chimeric antibodies can include those generated by immunization using purified recombinant TNFα or peptide fragments thereof comprising one or more epitopes.

An example of such a chimeric antibody is cA2 and antibodies which will competitively inhibit in vivo the binding to human TNFα of anti-TNFα murine mAb A2, chimeric mAb cA2, or an antibody having substantially the same specific binding characteristics, as well as fragments and regions thereof. Chimeric mAb cA2 has been described, for example, in U.S. application Ser. No. 08/192,093 (filed Feb. 4, 1994), U.S. application Ser. No. 08/192,102 (filed Feb. 4, 1994), U.S. application Ser. No. 08/192,861 (filed Feb. 4, 1994), and U.S. application Ser. No. 08/324,799 (filed Oct. 18, 1994), and by Le, J. et al. (International Publication No. WO 92/16553 (published Oct. 1, 1992)); Knight, D. M. et al. (Mol. Immunol. 30:1443-1453 (1993)); and Siegel, S. A. et al. (Cytokine 7(1):15-25 (1995)). These references are entirely incorporated herein by reference.

Chimeric A2 anti-TNF consists of the antigen binding variable region of the high-affinity neutralizing mouse anti-human TNF IgGl antibody, designated A2, and the constant regions of a human IgGl, kappa immunoglobulin. The human IgGl Fc region improves allogeneic antibody effector function, increases the circulating serum half-life and decreases the immunogenicity of the antibody. The avidity and epitope specificity of the chimeric A2 is derived from the variable region of the murine A2. Chimeric A2 neutralizes the cytotoxic effect of both natural and recombinant human TNF in a dose dependent manner. From binding assays of cA2 and recombinant human TNF, the affinity constant of cA2 was calculated to be 1.8×10⁹M⁻¹. Preferred methods for determining mAb specificity and affinity by competitive inhibition can be found in Harlow, et al., Antibodies: A Laboratory Manual, Cold Spring Harbor Laboratory Press, Cold Spring Harbor, N.Y., 1988; Colligan et al., eds., Current Protocols in Immunology, Greene Publishing Assoc. and Wiley Interscience, New York, (1992, 1993); Kozbor et al., Immunol. Today 4:72-79 (1983); Ausubel et al., eds. Current Protocols in Molecular Biology, Wiley Interscience, New York (1987, 1992, 1993); and Muller, Meth. Enzymol. 92:589-601 (1983), which references are entirely incorporated herein by reference.

As used herein, the term “antigen binding region” refers to that portion of an antibody molecule which contains the amino acid residues that interact with an antigen and confer on the antibody its specificity and affinity for the antigen. The antigen binding region includes the “framework” amino acid residues necessary to maintain the proper conformation of the antigen-binding residues. Generally, the antigen binding region will be of murine origin. In other embodiments, the antigen binding region can be derived from other animal species, such as sheep, rabbit, rat or hamster. Preferred sources for the DNA encoding such a non-human antibody include cell lines which produce antibody, preferably hybrid cell lines commonly known as hybridomas. In one embodiment, a preferred hybridoma is the A2 hybridoma cell line.

An “antigen” is a molecule or a portion of a molecule capable of being bound by an antibody which is additionally capable of inducing an animal to produce antibody capable of selectively binding to an epitope of that antigen. An antigen can have one or more than one epitope.

The term “epitope” is meant to refer to that portion of the antigen capable of being recognized by and bound by an antibody at one or more of the antibody's antigen binding region. Epitopes usually consist of chemically active surface groupings of molecules such as amino acids or sugar side chains and have specific three dimensional structural characteristics as well as specific charge characteristics. By “inhibiting and/or neutralizing epitope” is intended an epitope, which, when bound by an antibody, results in loss of biological activity of the molecule containing the epitope, in vivo or in vitro, more preferably in vivo, including binding of TNF to a TNF receptor. Epitopes of TNF have been identified within amino acids 1 to about 20, about 56 to about 77, about 108 to about 127 and about 138 to about 149. Preferably, the antibody binds to an epitope comprising at least about 5 amino acids of TNF within TNF residues from about 87 to about 108, about 59 to about 80, or a combination thereof. Generally, epitopes include at least about 5 amino acids and less than about 22 amino acids embracing or overlapping one or more of these regions.

Anti-TNF antibodies, and fragments, and variable regions thereof, that are recognized by, and/or binds with anti-TNF activity, these epitopes, block the action of TNFα without binding to the putative receptor binding locus as presented by Eck and Sprang (J. Biol. Chem. 264(29): 17595-17605 (1989) (amino acids 11-13, 37-42, 49-57 and 155-157 of hTNFα). Rathjen et al., International Publication No. WO 91/02078 (published Feb. 21, 1991), incorporated herein by reference, discloses TNF ligands which can bind additional epitopes of TNF.

Antibody Production Using Hybridomas

The techniques to raise antibodies to small peptide sequences that recognize and bind to those sequences in the free or conjugated form or when presented as a native sequence in the context of a large protein are well known in the art. Such antibodies can be produced by hybridoma or recombinant techniques known in the art.

Murine antibodies which can be used in the preparation of the antibodies useful in the methods and compositions of the present invention have also been described in U.S. application Ser. No. 08/192,093 (filed Feb. 4, 1994); U.S. application Ser. No. 08/192,102 (filed Feb. 4, 1994); U.S. application Ser. No. 08/192,861 (filed Feb. 4, 1994); U.S. application Ser. No. 08/324,799 (filed Oct. 18, 1994); Le, J. et al., International Publication No. WO 92/16553 (published Oct. 1, 1992); Rubin et al., EP 0218868 (published Apr. 22, 1987); Yone et al., EP 0288088 (published Oct. 26, 1988); Liang, et al., Biochem. Biophys. Res. Comm. 137:847-854 (1986); Meager, et al., Hybridoma 6:305-311 (1987); Fendly et al., Hybridoma 6:359-369 (1987); Bringman, et al., Hybridoma 6:489-507 (1987); Hirai, et al., J. Immunol. Meth. 96:57-62 (1987); and Möller, et al., Cytokine 2:162-169 (1990). The teachings of these references are entirely incorporated herein by reference.

The cell fusions are accomplished by standard procedures well known to those skilled in the field of immunology. Fusion partner cell lines and methods for fusing and selecting hybridomas and screening for mAbs are well known in the art. See, e.g., Ausubel infra, Harlow infra, and Colligan infra, the contents of which references are incorporated entirely herein by reference.

The TNFα-specific murine mAb useful in the methods and compositions of the present invention can be produced in large quantities by injecting hybridoma or transfectoma cells secreting the antibody into the peritoneal cavity of mice and, after appropriate time, harvesting the ascites fluid which contains a high titer of the mAb, and isolating the mAb therefrom. For such in vivo production of the mAb with a hybridoma (e.g., rat or human), hybridoma cells are preferably grown in irradiated or athymic nude mice. Alternatively, the antibodies can be produced by culturing hybridoma or transfectoma cells in vitro and isolating secreted mAb from the cell culture medium or recombinantly, in eukaryotic or prokaryotic cells.

In one embodiment, the antibody used in the methods and compositions of the present invention is a mAb which binds amino acids of an epitope of TNF recognized by A2, rA2 or cA2, produced by a hybridoma or by a recombinant host. In another embodiment, the antibody is a chimeric antibody which recognizes an epitope recognized by A2. In still another embodiment, the antibody is a chimeric antibody designated as chimeric A2 (cA2).

As examples of antibodies useful in the methods and compositions of the present invention, murine mAb A2 is produced by a cell line designated c134A. Chimeric antibody cA2 is produced by a cell line designated c168A.

“Derivatives” of the antibodies including fragments, regions or proteins encoded by truncated or modified genes to yield molecular species functionally resembling the immunoglobulin fragments are also useful in the methods and compositions of the present invention. The modifications include, but are not limited to, addition of genetic sequences coding for cytotoxic proteins such as plant and bacterial toxins. The fragments and derivatives can be produced from appropriate cells, as is known in the art. Alternatively, anti-TNF antibodies, fragments and regions can be bound to cytotoxic proteins or compounds in vitro, to provide cytotoxic anti-TNF antibodies which would selectively kill cells having TNF on their surface.

“Fragments” of the antibodies include, for example, Fab, Fab′, F(ab′)₂ and Fv. These fragments lack the Fc fragment of intact antibody, clear more rapidly from the circulation, and can have less non-specific tissue binding than an intact antibody (Wahl et al., J. Nucl. Med. 24:316-325 (1983)). These fragments are produced from intact antibodies using methods well known in the art, for example by proteolytic cleavage with enzymes such as papain (to produce Fab fragments) or pepsin (to produce F(ab′)₂ fragments).

Recombinant Expression of Anti-TNF Antibodies

Recombinant and/or chimeric murine-human or human-human antibodies that inhibit TNF can be produced using known techniques based on the teachings provided in U.S. application Ser. No. 08/192,093 (filed Feb. 4, 1994), U.S. application Ser. No. 08/192,102 (filed Feb. 4, 1994), U.S. application Ser. No. 08/192,861 (filed Feb. 4, 1994), U.S. application Ser. No. 08/324,799 (filed on Oct. 18, 1994) and Le, J. et al., International Publication No. WO 92/16553 (published Oct. 1, 1992), which references are entirely incorporated herein by reference. See, e.g., Ausubel et al., eds. Current Protocols in Molecular Biology, Wiley Interscience, New York (1987, 1992, 1993); and Sambrook et al. Molecular Cloning: A Laboratory Manual, Cold Spring Harbor Laboratory Press, New York (1989), the contents of which are entirely incorporated herein by reference. See also, e.g., Knight, D. M., et al., Mol. Immunol. 30:1443-1453 (1993); and Siegel, S. A., et al., Cytokine 7(1):15-25 (1995), the contents of which are entirely incorporated herein by reference.

The DNA encoding an anti-TNF antibody can be genomic DNA or cDNA which encodes at least one of the heavy chain constant region (Hc), the heavy chain variable region (Hc), the light chain variable region (Lv) and the light chain constant regions (Lc). A convenient alternative to the use of chromosomal gene fragments as the source of DNA encoding the murine V region antigen-binding segment is the use of cDNA for the construction of chimeric immunoglobulin genes, e.g., as reported by Liu et al. (Proc. Natl. Acad. Sci., USA 84:3439 (1987) and J. Immunology 139:3521 (1987)), which references are entirely incorporated herein by reference. The use of cDNA requires that gene expression elements appropriate for the host cell be combined with the gene in order to achieve synthesis of the desired protein. The use of cDNA sequences is advantageous over genomic sequences (which contain introns), in that cDNA sequences can be expressed in bacteria or other hosts which lack appropriate RNA splicing systems. An example of such a preparation is set forth below.

Because the genetic code is degenerate, more than one codon can be used to encode a particular amino acid. Using the genetic code, one or more different oligonucleotides can be identified, each of which would be capable of encoding the amino acid. The probability that a particular oligonucleotide will, in fact, constitute the actual XXX-encoding sequence can be estimated by considering abnormal base pairing relationships and the frequency with which a particular codon is actually used (to encode a particular amino acid) in eukaryotic or prokaryotic cells expressing an anti-TNF antibody or fragment. Such “codon usage rules” are disclosed by Lathe, et al., J. Mol. Biol. 183:1-12 (1985). Using the “codon usage rules” of Lathe, a single oligonucleotide, or a set of oligonucleotides, that contains a theoretical “most probable” nucleotide sequence capable of encoding anti-TNF variable or constant region sequences is identified.

Although occasionally an amino acid sequence can be encoded by only a single oligonucleotide, frequently the amino acid sequence can be encoded by any of a set of similar oligonucleotides. Importantly, whereas all of the members of this set contain oligonucleotides which are capable of encoding the peptide fragment and, thus, potentially contain the same oligonucleotide sequence as the gene which encodes the peptide fragment, only one member of the set contains the nucleotide sequence that is identical to the nucleotide sequence of the gene. Because this member is present within the set, and is capable of hybridizing to DNA even in the presence of the other members of the set, it is possible to employ the unfractionated set of oligonucleotides in the same manner in which one would employ a single oligonucleotide to clone the gene that encodes the protein.

The oligonucleotide, or set of oligonucleotides, containing the theoretical “most probable” sequence capable of encoding an anti-TNF antibody or fragment including a variable or constant region is used to identify the sequence of a complementary oligonucleotide or set of oligonucleotides which is capable of hybridizing to the “most probable” sequence, or set of sequences. An oligonucleotide containing such a complementary sequence can be employed as a probe to identify and isolate the variable or constant region anti-TNF gene (Sambrook et al., infra).

A suitable oligonucleotide, or set of oligonucleotides, which is capable of encoding a fragment of the variable or constant anti-TNF region (or which is complementary to such an oligonucleotide, or set of oligonucleotides) is identified (using the above-described procedure), synthesized, and hybridized by means well known in the art, against a DNA or, more preferably, a cDNA preparation derived from cells which are capable of expressing anti-TNF antibodies or variable or constant regions thereof. Single stranded oligonucleotide molecules complementary to the “most probable” variable or constant anti-TNF region peptide coding sequences can be synthesized using procedures which are well known to those of ordinary skill in the art (Belagaje, et al., J. Biol. Chem. 254:5765-5780 (1979); Maniatis, et al., In: Molecular Mechanisms in the Control of Gene Expression, Nierlich, et al., eds., Acad. Press, New York (1976); Wu, et al., Prog. Nucl. Acid Res. Molec. Biol. 21:101-141 (1978); Khorana, Science 203:614-625 (1979)). Additionally, DNA synthesis can be achieved through the use of automated synthesizers. Techniques of nucleic acid hybridization are disclosed by Sambrook et al., Molecular Cloning: A Laboratory Manual, Cold Spring Harbor Laboratory Press, New York (1989); and by Haynes, et al., in: Nucleic Acid Hybridization, A Practical Approach, IRL Press, Washington, D.C. (1985), which references are entirely incorporated herein by reference. Techniques such as, or similar to, those described above have successfully enabled the cloning of genes for human aldehyde dehydrogenases (Hsu, et al., Proc. Natl. Acad. Sci. USA 82:3771-3775 (1985)), fibronectin (Suzuki, et al., Bur. Mol. Biol. Organ. J. 4:2519-2524 (1985)), the human estrogen receptor gene (Walter, et al., Proc. Natl. Acad. Sci. USA 82:7889-7893 (1985)), tissue-type plasminogen activator (Pennica, et al., Nature 301:214-221 (1983)) and human placental alkaline phosphatase complementary DNA (Keun, et al., Proc. Natl. Acad. Sci. USA 82:8715-8719 (1985)).

In an alternative way of cloning a polynucleotide encoding an anti-TNF variable or constant region, a library of expression vectors is prepared by cloning DNA or, more preferably, cDNA (from a cell capable of expressing an anti-TNF antibody or variable or constant region) into an expression vector. The library is then screened for members capable of expressing a protein which competitively inhibits the binding of an anti-TNF antibody, such as A2 or cA2, and which has a nucleotide sequence that is capable of encoding polypeptides that have the same amino acid sequence as anti-TNF antibodies or fragments thereof. In this embodiment, DNA, or more preferably cDNA, is extracted and purified from a cell which is capable of expressing an anti-TNF antibody or fragment. The purified cDNA is fragmentized (by shearing, endonuclease digestion, etc.) to produce a pool of DNA or cDNA fragments. DNA or cDNA fragments from this pool are then cloned into an expression vector in order to produce a genomic library of expression vectors whose members each contain a unique cloned DNA or cDNA fragment such as in a lambda phage library, expression in prokaryotic cell (e.g., bacteria) or eukaryotic cells, (e.g., mammalian, yeast, insect or, fungus). See, e.g., Ausubel, infra; Harlow, infra; Colligan, infra; Nyyssonen et al. Bio/Technology 11:591-595 (1993); Marks et al., Bio/Technology 11:1145-1149 (October 1993). Once nucleic acid encoding such variable or constant anti-TNF regions is isolated, the nucleic acid can be appropriately expressed in a host cell, along with other constant or variable heavy or light chain encoding nucleic acid, in order to provide recombinant monoclonal antibodies that bind TNF with inhibitory activity. Such antibodies preferably include a murine or human anti-TNF variable region which contains a framework residue having complementarity determining residues which are responsible for antigen binding.

Human genes which encode the constant (C) regions of the chimeric antibodies, fragments and regions of the present invention can be derived from a human fetal liver library, by known methods. Human C region genes can be derived from any human cell including those which express and produce human immunoglobulins. The human CH region can be derived from any of the known classes or isotypes of human H chains, including gamma, μ, α, δ or ε, and subtypes thereof, such as G1, G2, G3 and G4. Since the H chain isotype is responsible for the various effector functions of an antibody, the choice of CH region will be guided by the desired effector functions, such as complement fixation, or activity in antibody-dependent cellular cytotoxicity (ADCC). Preferably, the CH region is derived from gamma 1 (IgG1), gamma 3 (IgG3), gamma 4 (IgG4), or μ (IgM). The human CL region can be derived from either human L chain isotype, kappa or lambda.

Genes encoding human immunoglobulin C regions are obtained from human cells by standard cloning techniques (Sambrook, et al., Molecular Cloning: A Laboratory Manual, 2nd Edition, Cold Spring Harbor Press, Cold Spring Harbor, N.Y. (1989) and Ausubel et al., eds., Current Protocols in Molecular Biology, Wiley Interscience, New York (1987-1993)). Human C region genes are readily available from known clones containing genes representing the two classes of L chains, the five classes of H chains and subclasses thereof. Chimeric antibody fragments, such as F(ab′)₂ and Fab, can be prepared by designing a chimeric H chain gene which is appropriately truncated. For example, a chimeric gene encoding an H chain portion of an F(ab′)₂ fragment would include DNA sequences encoding the CH1 domain and hinge region of the H chain, followed by a translational stop codon to yield the truncated molecule.

Generally, the murine, human and chimeric antibodies, fragments and regions are produced by cloning DNA segments encoding the H and L chain antigen-binding regions of a TNF-specific antibody, and joining these DNA segments to DNA segments encoding CH and CL regions, respectively, to produce murine, human or chimeric immunoglobulin-encoding genes. Thus, in a preferred embodiment, a fused chimeric gene is created which comprises a first DNA segment that encodes at least the antigen-binding region of non-human origin, such as a functionally rearranged V region with joining (J) segment, linked to a second DNA segment encoding at least a part of a human C region.

Therefore, cDNA encoding the antibody V and C regions and the method of producing a chimeric antibody can involve several steps, outlined below:
1. isolation of messenger RNA (mRNA) from the cell line producing an anti-TNF antibody and from optional additional antibodies supplying heavy and light constant regions; cloning and cDNA production therefrom;
2. preparation of a full length cDNA library from purified mRNA from which the appropriate V and/or C region gene segments of the L and H chain genes can be: (i) identified with appropriate probes, (ii) sequenced, and (iii) made compatible with a C or V gene segment from another antibody for a chimeric antibody;
3. Construction of complete H or L chain coding sequences by linkage of the cloned specific V region gene segments to cloned C region gene, as described above;
4. Expression and production of L and H chains in selected hosts, including prokaryotic and eukaryotic cells to provide murine-murine, human-murine or human-human antibodies.

One common feature of all immunoglobulin H and L chain genes and their encoded mRNAs is the J region. H and L chain J regions have different sequences, but a high degree of sequence homology exists (greater than 80%) among each group, especially near the C region. This homology is exploited in this method and consensus sequences of H and L chain J regions can be used to design oligonucleotides for use as primers for introducing useful restriction sites into the J region for subsequent linkage of V region segments to human C region segments.

C region cDNA vectors prepared from human cells can be modified by site-directed mutagenesis to place a restriction site at the analogous position in the human sequence. For example, one can clone the complete human kappa chain C (Ck) region and the complete human gamma-1 C region (C gamma-1). In this case, the alternative method based upon genomic C region clones as the source for C region vectors would not allow these genes to be expressed in bacterial systems where enzymes needed to remove intervening sequences are absent. Cloned V region segments are excised and ligated to L or H chain C region vectors. Alternatively, the human C gamma-1 region can be modified by introducing a termination codon thereby generating a gene sequence which encodes the H chain portion of an Fab molecule. The coding sequences with linked V and C regions are then transferred into appropriate expression vehicles for expression in appropriate hosts, prokaryotic or eukaryotic.

Two coding DNA sequences are said to be “operably linked” if the linkage results in a continuously translatable sequence without alteration or interruption of the triplet reading frame. A DNA coding sequence is operably linked to a gene expression element if the linkage results in the proper function of that gene expression element to result in expression of the coding sequence.

Expression vehicles include plasmids or other vectors. Preferred among these are vehicles carrying a functionally complete human CH or CL chain sequence having appropriate restriction sites engineered so that any VH or VL chain sequence with appropriate cohesive ends can be easily inserted therein. Human CH or CL chain sequence-containing vehicles thus serve as intermediates for the expression of any desired complete H or L chain in any appropriate host.

A chimeric antibody, such as a mouse-human or human-human, will typically be synthesized from genes driven by the chromosomal gene promoters native to the mouse H and L chain V regions used in the constructs; splicing usually occurs between the splice donor site in the mouse J region and the splice acceptor site preceding the human C region and also at the splice regions that occur within the human C, region; polyadenylation and transcription termination occur at native chromosomal sites downstream of the human coding regions.

A nucleic acid sequence encoding at least one anti-TNF antibody fragment may be recombined with vector DNA in accordance with conventional techniques, including blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases. Techniques for such manipulations are disclosed, e.g., by Ausubel, supra, Sambrook, supra, entirely incorporated herein by reference, and are well known in the art.

A nucleic acid molecule, such as DNA, is “capable of expressing” a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene expression as anti-TNF peptides or antibody fragments in recoverable amounts. The precise nature of the regulatory regions needed for gene expression may vary from organism to organism and is well known in the analogous art. See, e.g., Sambrook et al., Molecular Cloning: A Laboratory Manual, Cold Spring Harbor Laboratory Press, New York (1989); and Ausubel, eds., Current Protocols in Molecular Biology, Wiley Interscience, New York (1987, 1993).

Many vector systems are available for the expression of cloned anti-TNF peptide H and L chain genes in mammalian cells (see Glover, ed., DNA Cloning, Vol. II, pp. 143-238, IRL Press, Washington, D.C., 1985). Different approaches can be followed to obtain complete H2L2 antibodies. It is possible to co-express H and L chains in the same cells to achieve intracellular association and linkage of H and L chains into complete tetrameric H2L2 antibodies. The co-expression can occur by using either the same or different plasmids in the same host. Genes for both H and L chains can be placed into the same plasmid, which is then transfected into cells, thereby selecting directly for cells that express both chains. Alternatively, cells can be transfected first with a plasmid encoding one chain, for example the L chain, followed by transfection of the resulting cell line with an H chain plasmid containing a second selectable marker. Cell lines producing H2L2 molecules via either route could be transfected with plasmids encoding additional copies of peptides, H, L, or H plus L chains in conjunction with additional selectable markers to generate cell lines with enhanced properties, such as higher production of assembled H2L2 antibody molecules or enhanced stability of the transfected cell lines.

TNF Receptor Molecules

TNF receptor molecules useful in the methods and compositions of the present invention are those that bind TNF with high affinity (see, e.g., Feldmann et al., International Publication No. WO 92/07076 (published Apr. 30, 1992); Schall et al., Cell 61:361-370 (1990); and Loetscher et al., Cell 61:351-359 (1990), which references are entirely incorporated herein by reference) and possess low immunogenicity. In particular, the 55 kDa (p55 TNF-R) and the 75 kDa (p75 TNF-R) TNF cell surface receptors are useful in the present invention. Truncated forms of these receptors, comprising the extracellular domains (ECD) of the receptors or functional portions thereof (see, e.g., Corcoran et al., Eur. J. Biochem. 223:831-840 (1994)), are also useful in the present invention. Truncated forms of the TNF receptors, comprising the ECD, have been detected in urine and serum as 30 kDa and 40 kDa TNF inhibitory binding proteins (Engelmann, H. et al., J. Biol. Chem. 265:1531-1536 (1990)). TNF receptor multimeric molecules and TNF immunoreceptor fusion molecules, and derivatives and fragments or portions thereof, are additional examples of TNF receptor molecules which are useful in the methods and compositions of the present invention. The TNF receptor molecules which can be used in the invention are characterized by their ability to treat patients for extended periods with good to excellent alleviation of symptoms and low toxicity. Low immunogenicity and/or high affinity, as well as other undefined properties, may contribute to the therapeutic results achieved.

TNF receptor multimeric molecules useful in the present invention comprise all or a functional portion of the ECD of two or more TNF receptors linked via one or more polypeptide linkers or other nonpeptide linkers, such as polyethylene glycol (PEG). The multimeric molecules can further comprise a signal peptide of a secreted protein to direct expression of the multimeric molecule. These multimeric molecules and methods for their production have been described in U.S. application Ser. No. 08/437,533 (filed May 9, 1995), the content of which is entirely incorporated herein by reference.

TNF immunoreceptor fusion molecules useful in the methods and compositions of the present invention comprise at least one portion of one or more immunoglobulin molecules and all or a functional portion of one or more TNF receptors. These immunoreceptor fusion molecules can be assembled as monomers, or hetero- or homo-multimers. The immunoreceptor fusion molecules can also be monovalent or multivalent. An example of such a TNF immunoreceptor fusion molecule is TNF receptor/IgG fusion protein.

TNF immunoreceptor fusion molecules and methods for their production have been described in the art (Lesslauer et al., Eur. J. Immunol. 21:2883-2886 (1991); Ashkenazi et al., Proc. Natl. Acad. Sci. USA 88:10535-10539 (1991); Peppel et al., J. Exp. Med. 174:1483-1489 (1991); Kolls et al., Proc. Natl. Acad. Sci. USA 91:215-219 (1994); Butler et al., Cytokine 6(6):616-623 (1994); Baker et al., Eur. J. Immunol. 24:2040-2048 (1994); Beutler et al., U.S. Pat. No. 5,447,851; and U.S. application Ser. No. 08/442,133 (filed May 16, 1995), which references are entirely incorporated herein by reference). Methods for producing immunoreceptor fusion molecules can also be found in Capon et al., U.S. Pat. No. 5,116,964; Capon et al., U.S. Pat. No. 5,225,538; and Capon et al., Nature 337:525-531 (1989), which references are entirely incorporated herein by reference.

Derivatives, fragments, regions and functional portions of the TNF receptor molecules functionally resemble the TNF receptor molecules that can be used in the present invention (i.e., they bind TNF with high affinity and possess low immunogenicity). A functional equivalent or derivative of the TNF receptor molecule refers to the portion of the TNF receptor molecule, or the portion of the TNF receptor molecule sequence which encodes the TNF receptor molecule, that is of sufficient size and sequences to functionally resemble the TNF receptor molecules that can be used in the present invention (i.e., bind TNF with high affinity and possess low immunogenicity). A functional equivalent of the TNF receptor molecule also includes modified TNF receptor molecules that functionally resemble the TNF receptor molecules that can be used in the present invention (i.e., bind TNF with high affinity and possess low immunogenicity). For example, a functional equivalent of the TNF receptor molecule can contain a “SILENT” codon or one or more amino acid substitutions, deletions or additions (e.g., substitution of one acidic amino acid for another acidic amino acid; or substitution of one codon encoding the same or different hydrophobic amino acid for another codon encoding a hydrophobic amino acid). See Ausubel et al., Current Protocols in Molecular Biology, Greene Publishing Assoc. and Wiley-Interscience, New York (1989).

IL-12 Antagonists

As used herein, an “IL-12 antagonist” decreases, blocks, inhibits, abrogates or interferes with IL-12 activity, synthesis or receptor signalling in vivo. IL-12 antagonists include anti-IL-12 antibodies, receptor molecules which bind specifically to IL-12, IL-12 receptor antagonists, IL-12 p40 homodimers, IL-12 p35 homodimers, and other appropriate peptides and small molecules. IL-12 p40 homodimers are described in the art (see, e.g., Ling et al., J. Immunol. 154(1):116-127 (1995); and Gillessen et al., Eur. J. Immunol. 25(1):200-206 (1995), which references are entirely incorporated herein by reference). IL-12 antagonists also include agents which decrease, inhibit, block, abrogate or interfere with IL-12 production, such as compounds (e.g. drugs and other agents, including antibodies) which inhibit, block, abrogate or interfere with CD40 or its ligands; adrenergic agonists, such as, but not limited to, salbutamol; and cytokines, such as IL-4, IL-10, IL-13 and TGFβ. IL-12 antagonists also include agents (e.g., drugs and other agents) which decrease, abrogate, block, inhibit or interfere with IL-12 receptor signalling.

Anti-IL-12 Antibodies

Anti-IL-12 antibodies useful in the present invention include polyclonal, monoclonal, chimeric, humanized, resurfaced and recombinant antibodies and fragments thereof which are characterized by high affinity binding to IL-12 and low toxicity (including HAMA and/or HACA response). In particular, an antibody where the individual components, such as the variable region, constant region and framework, individually and/or collectively possess low immunogenicity is useful in the present invention. The antibodies which can be used in the invention are characterized by their ability to treat patients for extended periods with good to excellent alleviation of symptoms and low toxicity. Low immunogenicity and/or high affinity, as well as other undefined properties, may contribute to the therapeutic results achieved.

Techniques described herein for producing anti-TNF antibodies can be employed in producing anti-IL-12 antibodies that can be used in the present invention.

Monoclonal antibodies reactive with IL-12 can be produced using somatic cell hybridization techniques (Kohler and Milstein, Nature 256: 495-497 (1975)) or other techniques. In a typical hybridization procedure, a crude or purified protein or peptide comprising at least a portion of IL-12 can be used as the immunogen. An animal is vaccinated with the immunogen to obtain anti-IL-12 antibody-producing spleen cells. The species of animal immunized will vary depending on the species of monoclonal antibody desired. The antibody producing cell is fused with an immortalizing cell (e.g., myeloma cell) to create a hybridoma capable of secreting anti-IL-12 antibodies. The unfused residual antibody-producing cells and immortalizing cells are eliminated. Hybridomas producing desired antibodies are selected using conventional techniques and the selected hybridomas are cloned and cultured.

Polyclonal antibodies can be prepared by immunizing an animal with a crude or purified protein or peptide comprising at least a portion of IL-12. The animal is maintained under conditions whereby antibodies reactive with IL-12 are produced. Blood is collected from the animal upon reaching a desired titre of antibodies. The serum containing the polyclonal antibodies (antisera) is separated from the other blood components. The polyclonal antibody-containing serum can optionally be further separated into fractions of particular types of antibodies (e.g., IgG, IgM).

Examples of anti-IL-12 antibodies that can be used in the present invention are described in the art (see, e.g., Wilkinson et al., J. Immunol. Methods 189:15-24 (1996); Tripp et al., J. Immunol. 152:1883-1887 (1994); Trinchieri et al., Blood 84:4008-4027 (1994); Wysocka et al., Eur. J. Immunol. 25:672-676 (1995); Neurath et al., J. Exp. Med. 182:1281-1290 (1995); Chizzonite et al., J. Immunol. 147(5):1548-1556 (1991); Ozmen et al., J. Exp. Med. 180:907-915 (1994); D'Andrea et al., J. Exp. Med. 176:1387-1398 (1992); Ozmen et al., J. Exp. Med. 180:907-915 (1994); Riemann et al., J. Immunol. 156(5):1799-1803 (1996); Gazzinelli et al., J. Immunol. 153(6):2533-2543 (1994); Jones, Scand. J. Immunol. 43(1):64-72 (1996); Sypek et al., J. Exp. Med. 177:1797-1802 (1993); and Valiante et al., Cell Immunol. 145(1):187-198 (1992), which references are entirely incorporated herein by reference).

IL-12 Receptor Molecules

IL-12 receptor molecules useful in the present invention bind specifically to IL-12 and possess low immunogenicity. Preferably, the IL-12 receptor molecule is characterized by high affinity binding to IL-12. IL-12 receptor molecules include IL-12 receptors, IL-12 receptor multimeric molecules and IL-12 immunoreceptor fusion molecules, and derivatives and fragments or portions thereof. An example of an IL-12 receptor molecule is described by Chua et al. (U.S. Application No. 5,536,657; and Chua et al., J. Immunol. 153:128-136 (1994), the teachings of which are entirely incorporated herein by reference). The IL-12 receptor molecules which can be used in the invention are characterized by their ability to treat patients for extended periods with good to excellent alleviation of symptoms and low toxicity. Low immunogenicity and/or high affinity, as well as other undefined properties, may contribute to the therapeutic results achieved.

IL-12 receptor multimeric molecules can comprise all or a functional portion of two or more IL-12 receptors linked via one or more linkers. IL-12 receptor multimeric molecules can further comprise a signal peptide of a secreted protein to direct expression of the multimeric molecule.

IL-12 immunoreceptor fusion molecules can comprise at least one portion of one or more immunoglobulin molecules and all or a functional portion of one or more IL-12 receptors. IL-12 immunoreceptor fusion molecules can be assembled as monomers, or hetero- or homo-multimers. IL-12 immunoreceptor fusion molecules can also be monovalent or multivalent.

Derivatives, fragments, regions and functional portions of the IL-12 receptor molecules functionally resemble the IL-12 receptor molecules that can be used in the present invention (i.e., they bind specifically to IL-12 and possess low immunogenicity). A functional equivalent or derivative of the IL-12 receptor molecule refers to the portion of the IL-12 receptor molecule, or the portion of the IL-12 receptor molecule sequence which encodes the IL-12 receptor molecule, that is of sufficient size and sequences to functionally resemble the IL-12 receptor molecules that can be used in the present invention (i.e., bind specifically to IL-12 and possess low immunogenicity). A functional equivalent of the IL-12 receptor molecule also includes modified IL-12 receptor molecules that functionally resemble the IL-12 receptor molecules that can be used in the present invention (i.e., bind specifically to IL-12 and possess low immunogenicity). For example, a functional equivalent of the IL-12 receptor molecule can contain a “SILENT” codon or one or more amino acid substitutions, deletions or additions (e.g., substitution of one acidic amino acid for another acidic amino acid; or substitution of one codon encoding the same or different hydrophobic amino acid for another codon encoding a hydrophobic amino acid). See Ausubel et al., Current Protocols in Molecular Biology, Greene Publishing Assoc. and Wiley-Interscience, New York (1989).

Techniques described herein for producing TNF receptor molecules can be employed in producing IL-12 receptor molecules that can be used in the present invention.

Administration

TNF antagonists, IL-12 antagonists, and the compositions of the present invention can be administered to an individual in a variety of ways. The routes of administration include intradermal, transdermal (e.g., in slow release polymers), intramuscular, intraperitoneal, intravenous, subcutaneous, oral, topical, epidural, buccal, rectal, vaginal and intranasal routes. Any other route of administration can be used, for example, infusion or bolus injection, absorption through epithelial or mucocutaneous linings, or by gene therapy wherein a DNA molecule encoding the therapeutic protein or peptide is administered to the patient, e.g., via a vector, which causes the protein or peptide to be expressed and secreted at therapeutic levels in vivo. In addition, the TNF antagonists, IL-12 antagonists and compositions of the present invention can be administered together with other components of biologically active agents, such as pharmaceutically acceptable surfactants (e.g., glycerides), excipients (e.g., lactose), carriers, diluents and vehicles. If desired, certain sweetening, flavoring and/or coloring agents can also be added.

The TNF antagonists and IL-12 antagonists can be administered prophylactically or therapeutically to an individual. TNF antagonists can be administered prior to, simultaneously with (in the same or different compositions) or sequentially with the administration of an IL-12 antagonist.

For parenteral (e.g., intravenous, subcutaneous, intramuscular) administration, TNF antagonists, IL-12 antagonists and the compositions of the present invention can be formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and nonaqueous vehicles such as fixed oils can also be used. The vehicle or lyophilized powder can contain additives that maintain isotonicity (e.g., sodium chloride, mannitol) and chemical stability (e.g., buffers and preservatives). The formulation is sterilized by commonly used techniques.

Suitable pharmaceutical carriers are described in Remington's Pharmaceutical Sciences, A. Osol, a standard reference text in this field of art.

For example, a parenteral composition suitable for administration by injection is prepared by dissolving 1.5% by weight of active ingredient in 0.9% sodium chloride solution.

TNF antagonists and IL-12 antagonists are co-administered simultaneously or sequentially in therapeutically effective amounts; the compositions of the present invention are administered in a therapeutically effective amount. As used herein, a “therapeutically effective amount” is such that co-administration of TNF antagonist and IL-12 antagonist, or administration of a composition of the present invention, results in inhibition of the biological activity of TNF relative to the biological activity of TNF when therapeutically effective amounts of TNF antagonist and IL-12 antagonist are not co-administered, or relative to the biological activity of TNF when a therapeutically effective amount of the composition is not administered. A therapeutically effective amount is that amount of TNF antagonist and IL-12 antagonist necessary to significantly reduce or eliminate symptoms associated with a particular TNF-mediated disease. As used herein, a therapeutically effective amount is not an amount such that administration of the TNF antagonist alone, or administration of the IL-12 antagonist alone, must necessarily result in inhibition of the biological activity of TNF or in immunosuppressive activity.

Once a therapeutically effective amount has been administered, a maintenance amount of TNF antagonist alone, of IL-12 antagonist alone, or of a combination of TNF antagonist and IL-12 antagonist can be administered to the individual. A maintenance amount is the amount of TNF antagonist, IL-12 antagonist, or combination of TNF antagonist and IL-12 antagonist necessary to maintain the reduction or elimination of symptoms achieved by the therapeutically effective dose. The maintenance amount can be administered in the form of a single dose, or a series or doses separated by intervals of days or weeks.

The dosage administered to an individual will vary depending upon a variety of factors, including the pharmacodynamic characteristics of the particular antagonists, and its mode and route of administration; size, age, health, sex, body weight and diet of the recipient; nature and extent of symptoms of the disease being treated, kind of concurrent treatment, frequency of treatment, and the effect desired. In vitro and in vivo methods of determining the inhibition of TNF in an individual are well known to those of skill in the art. Such in vitro assays can include a TNF cytotoxicity assay (e.g., the WEHI assay or a radioimmunoassay, ELISA). In vivo methods can include rodent lethality assays, primate pathology model systems (see, e.g., Mathison et al., J. Clin. Invest. 81: 1925-1937 (1988); Beutler et al., Science 229: 869-871 (1985); Tracey et al., Nature 330: 662-664 (1987); Shimamoto et al., Immunol. Lett. 17: 311-318 (1988); Silva et al., J. Infect. Dis. 162: 421-427 (1990); Opal et al., J. Infect. Dis. 161: 1148-1152 (1990); and Hinshaw et al., Circ. Shock 30: 279-292 (1990)) and/or rodent models of arthritis (Williams et al., Proc. Natl. Acad. Sci. USA 89:9784-9788 (1992)). In patients with rheumatoid arthritis, TNFα blockade can be monitored by monitoring IL-6 and C-reactive protein levels (Elliott et al., Arthritis Rheum. 36:1681-1690 (1993)).

TNF antagonists and IL-12 antagonists can be co-administered in single or multiple doses depending upon factors such as nature and extent of symptoms, kind of concurrent treatment and the effect desired. Thus, other therapeutic regimens or agents (e.g., multiple drug regimens) can be used in combination with the therapeutic co-administration of TNF antagonists and IL-12 antagonists. Adjustment and manipulation of established dosage ranges are well within the ability of those skilled in the art.

Usually a daily dosage of active ingredient can be about 0.01 to 100 milligrams per kilogram of body weight. Ordinarily 1 to 40 milligrams per kilogram per day given in divided doses 1 to 6 times a day or in sustained release form is effective to obtain desired results. Second or subsequent administrations can be administered at a dosage which is the same, less than or greater than the initial or previous dose administered to the individual.

A second or subsequent administration is preferably during or immediately prior to relapse or a flare-up of the disease or symptoms of the disease. For example, the second and subsequent administrations can be given between about one day to 30 weeks from the previous administration. Two, three, four or more total administrations can be delivered to the individual, as needed.

Dosage forms (composition) suitable for internal administration generally contain from about 0.1 milligram to about 500 milligrams of active ingredient per unit. In these pharmaceutical compositions the active ingredient will ordinarily be present in an amount of about 0.5-95% by weight based on the total weight of the composition.

The present invention will now be illustrated by the following examples, which are not intended to be limiting in any way.

### EXAMPLE

### Example Treatment of Induced Arthritis in a Murine Model Using Anti-TNF Antibody and Anti-IL-12 Antibody

The murine model of type II collagen induced arthritis (CIA) has similarities to rheumatoid arthritis (RA) in its marked MHC class II predisposition, as well as in histology, immunohistology, and erosions of cartilage and bone. Furthermore, there is a good correlation of its therapeutic response with that of human rheumatoid arthritis. For example, in both diseases anti-TNF antibody has beneficial effects (Williams, R. O. et al., Proc. Natl. Acad. Sci. USA 89:9784-9788 (1992); Elliott, M. J. et al., Arthritis & Rheumatism 36:1681-1690 (1993)). Thus, the animal model serves as a good approximation to human disease.

The model of rheumatoid arthritis used herein, i.e., the type II collagen induced arthritis in the DBA/1 mouse, is described by Williams, R. O. et al. (Proc. Natl. Acad. Sci. USA 89:9784-9788 (1992)). Type II collagen (CII) was purified from bovine hyaline cartilage by limited pepsin solubilization and salt fractionation as described by Miller (Biochemistry 11:4903-4909 (1972)).

Anti-IL-12 Antibody

The anti-IL-12 p40 antibody (clone 17.8) is a rat anti-mouse IgG2a monoclonal antibody provided by Giorgio Trinchieri at the Wistar Institute (Philadelphia, Pa.) (Trinchieri et al., Blood 84:4008-4027 (1994); Wysocka et al., Eur. J. Immunol. 25: 672-676 (1995); and Neurath et al., J. Exp. Med. 182:1281-1290 (1995), which references are entirely incorporated herein by reference).

Anti-TNF Antibody

The anti-TNF antibody cV1q was constructed by Centocor, Inc. (Malvern, Pa.). Hybridoma cells secreting the rat anti-murine TNF antibody V1q were from Peter Krammer at the German Cancer Research Center, Heidelberg, Germany (Echtenacher et al., J. Immunol. 145:3762-3766 (1990)). Genes encoding the variable regions of the heavy and light chains of the anti-TNF antibody V1q were cloned. The cloned heavy chain was inserted into four different gene expression vectors to encode cV1q heavy chain with either a human IgG1, human IgG3, murine IgG1 or murine IgG2a constant region. The V1q light chain gene was inserted into other expression vectors to encode either a human kappa or a murine kappa light chain constant region.

SP2/O myeloma cells were transfected with the different heavy and light chain gene constructs. Cell clones producing chimeric V1q (cV1q) were identified by assaying cell supernatant for human or murine IgG using standard ELISA assays. High-producing clones were subcloned to obtain homogenous cell lines. The murine IgG1 and IgG2a versions are referred to as C257A and C258A, respectively. cV1q was purified from cell supernatant by protein A chromatography.

cV1q was characterized by measuring its affinity for soluble murine TNF, testing its ability to protect WEHI cells from murine TNF cytotoxicity, examining its ability to neutralize or bind murine lymphotoxin, comparing the ability of the murine IgG1 and IgG2a versions to trigger complement-mediated lysis of cells expressing recombinant transmembrane murine TNF, and examining the ability of the human IgG1 version to protect mice from lethal doses of LPS (endotoxin). The murine IgG1 version of cV1q was used in the following experimental procedure.

Experimental Procedure

Male DBA/1 mice were immunized intradermally at the base of the tail at 8-12 weeks of age with 100 μg type II collagen emulsified in Freund's complete adjuvant (Difco Laboratories, Detroit, Mich.). From day 15 after immunization onwards, mice were examined daily for onset of disease using two clinical parameters: paw swelling and clinical score.

Paw-swelling was assessed by measuring the thickness of the affected hindpaws with calipers.

Clinical score was assessed on the following scale: 0=normal; 1=slight swelling and erythema; 2=pronounced edema; and 3=joint rigidity. Each limb was graded, giving a maximum clinical score of 12 per mouse.

Day one of arthritis was considered to be the day that erythema and/or swelling in one or more limbs was first observed. Arthritis became clinically evident around 18 to 30 days (average 23 days) after immunization with type II collagen. After the onset of clinically evident arthritis, two groups of mice (Group 1 and Group 2; 9 mice per group) were subjected to treatment with one of the following therapies: 500 μg/ml anti-IL-12 p40 antibody (clone 17.8), injected intra-peritoneally once every other day (days 1, 3, 5, 7 and 9); 300 μg/ml anti-TNF antibody cV1q, injected intra-peritoneally once every other day (days 1, 3, 5, 7 and 9); 500 μg/ml anti-IL-12 p40 antibody (clone 17.8), injected intra-peritoneally once every other day (days 1, 3, 5, 7 and 9) in conjunction with 300 μg/ml anti-TNF antibody cV1q, injected intra-peritoneally once every other day (days 1, 3, 5, 7 and 9); or phosphate-buffered saline (PBS; control), injected intra-peritoneally once every other day.

Arthritis in the mice was monitored using paw-swelling and clinical score over a 10 day period, after which the mice were sacrificed and joints were processed for histology. The Mann-Whitney U test was applied to all clinical results to compare non-parametric data for statistical significance. The chi-square test was applied for analysis of histological data.

Paw-Swelling

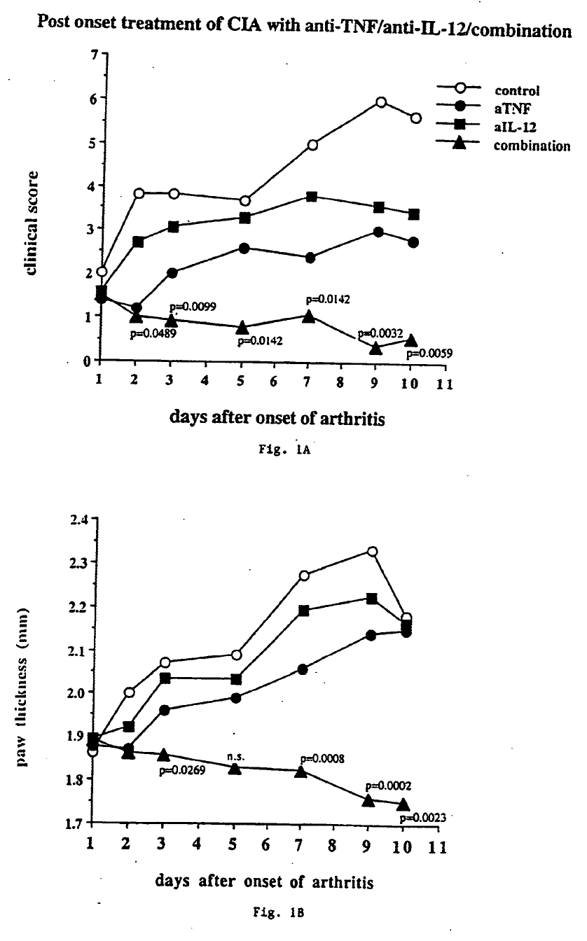

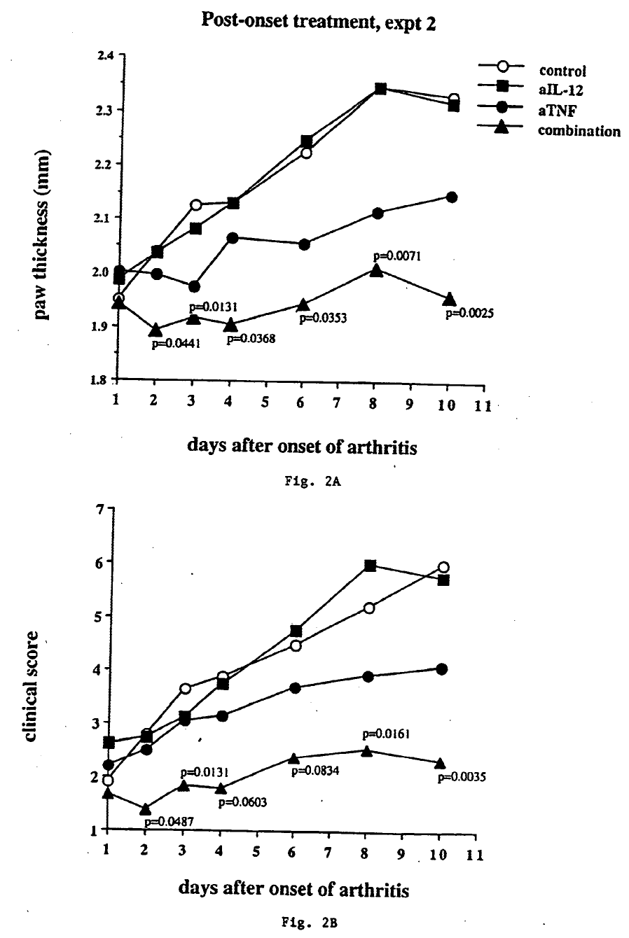

Treatment with a combination of anti-TNF antibody and anti-IL-12 antibody resulted in a highly significant and sustained reduction in paw-swelling over the treatment period. Results are shown in FIG. 1B (Group 1) and FIG. 2A (Group 2). p-values are given for the combination treated mice versus the PBS treated mice (Mann-Whitney U test).

Clinical Score

Clinical score results are presented in FIG. 1A (Group 1) and FIG. 2B (Group 2) and confirm that treatment with a combination of anti-TNF antibody and anti-IL-12 antibody significantly ameliorated disease. That is, when anti-TNF antibody and anti-IL-12 antibody are administered together, there was a highly significant reduction in the severity of arthritis. p-values are given for the combination treated mice versus the PBS treated mice (Mann-Whitney U test).

Histology

Hindpaws were removed post mortem on the tenth day of arthritis, fixed in formalin and decalcified in 5% EDTA. Paws were then embedded in paraffin, sectioned and stained with hematoxylin and eosin. Arthritic changes in the ankle, the metatarsophalangeal joints, the proximal interphalangeal and the distal interphalangeal joints were scored blindly as mild (=mild synovial hyperplasis); moderate (=pannus formation and erosions limited to the cartilage-pannus junction); or severe (=extended bone and cartilage erosions with loss of joint architecture).

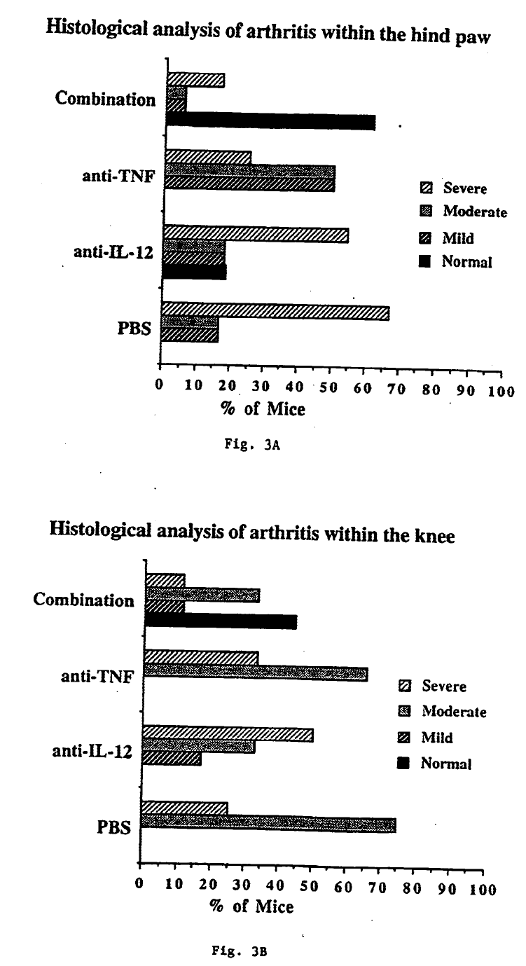

Results are presented in FIGS. 3A and 3B. Significantly more joints in the hind paws were scored as “normal” or “mild” in the mice treated with a combination of anti-TNF antibody and anti-IL-12 antibody compared to the mice treated with PBS (FIG. 3A; p<0.01, chi-square test). Similar results were observed in the knee joints (FIG. 3B; p=0.05).

Anti-Collagen IgG Levels

Mice were bled post mortem, i.e., after 10 days of arthritis. Serum levels of anti-CII antibodies (IgG1 and IgG2a subclasses) were measured by modification of an enzyme-linked immunosorbent assay (ELISA) as described in detail for the detection of human IgG (Williams et al., Proc. Natl. Acad. Sci. USA 89:9784-9788 (1992)). Briefly, microtitre plates were coated with native bovine type II collagen (2 μg/ml), blocked, then incubated with serially diluted test sera. Bound IgG was detected by incubation with alkaline phosphatase-conjugated sheep anti-mouse IgG1 or IgG2a (Binding Site, Birmingham, UK), followed by substrate (dinitrophenyl phosphate). Plates were washed between steps with 0.01% Tween-PBS. Optical densities were read at 405 nm.

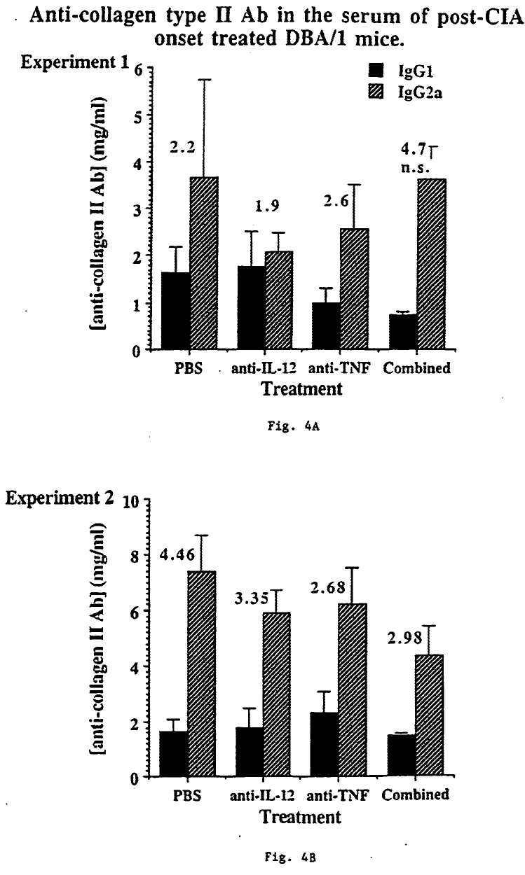

The serum levels of IgG1 and IgG2a anti-CII antibodies are shown in FIG. 4A (Group 1) and FIG. 4B (Group 2) for each treatment regimen. The ratio of serum IgG2a/IgG1 subclasses is given for each treatment regimen. Serum levels of IgG1 and IgG2a anti-CII antibodies, as well as the ratio of serum IgG2a/IgG1 subclasses, were not significantly altered within the 10 day treatment period by anti-TNF antibody alone, anti-IL-12 antibody alone or anti-TNF antibody plus anti-IL-12 antibody.

### SUMMARY

Treatment of CIA with a combination of anti-TNF antibody and anti-IL-12 antibody resulted in dramatic suppression of joint inflammation, as measured by swelling and redness of the hind paws and by decreased spreading of the disease, as reflected in the low clinical scores. Clinical results were confirmed by the histological data, which showed a marked protection against joint destruction in the mice treated with a combination of anti-TNF antibody and anti-IL-12 antibody. This was demonstrated not only in the hind paws but also in the knee joints.

Amelioration of arthritis was not accompanied by changes in total IgG anti-CII antibody levels or in the serum IgG2a/IgG1 ratio, probably due to the long half-life of serum antibodies relative to the duration of the experiment.

The magnitude of the benefit obtained by combining anti-IL-12 and anti-TNF antibodies was considerable, unlike treatment with anti-IL-12 antibody alone. The results show a synergy between anti-TNF antibody and anti-IL-12 antibody in blocking progression of CIA after onset of disease. This suggests that therapy with a combination of anti-TNF antibody and anti-IL-12 antibody is valuable in the treatment of rheumatoid arthritis.

### EQUIVALENTS

Those skilled in the art will know, or be able to ascertain, using no more than routine experimentation, many equivalents to the specific embodiments of the invention described herein. These and all other equivalents are intended to be encompassed by the following claims.

1-32. (canceled)

33. A method of treating an individual in need of treatment for arthritis which comprises co-administering to the individual an anti-tumor necrosis factor antibody or a fragment thereof which binds to tumor necrosis factor and an anti-interleukin-12 antibody or a fragment thereof which binds to interleukin-12, the amounts of such antibodies being effective to suppress clinical symptoms associated with arthritis in an individual.
